SEQUENCE LISTING (12 sequences)

SEQ ID NO: 1 - DNA, 20 bp, synthetic construct
  source 1..20: mol_type = other DNA
aatgagcagaatctatatgg

SEQ ID NO: 2 - DNA, 5485 bp, synthetic construct
  source 1..5485: mol_type = other DNA
acataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgatttgataccgcgggccctaaacgcgtaccaacgtgaaaaaattattattcgcataacttcgtatagcatacattatacgaagttatcctcagcaccatggctagcggcagcctcggagtttgaatagatagaataaaatatctttattttcattccatctgtgtgttggttttttgtgtgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcaggctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgcctcagctaattgagtaggggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccgtggaattcgtaaatgaattttctgtatgaggtcgcgatgaataaatgaaagcttgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagctgatccggaacccttaatataacttcgtatagcatacattatacgaagttattaggtccctcgacctgcagcccaagctagatcgaattcggccggccttcacgagccgccaccctcgaggccaccatggattccaaacaccagtgtgtaaagctaaatgatggccacttcatgcctgtattgggatttggcacctatgcacctccagaggttccgagaagtaaagctttggaggtcacaaaattagcaatagaagctgggttccgccatatagattctgctcatttatacaataatgaggagcaggttggactggccatccgaagcaagattgcagatggcagtgtgaagagagaagacatattctacacttcaaagctttggtccacttttcatcgaccagagttggtccgaccagccttggaaaactcactgaagaaagctcaattggactatgttgacctctatcttattcattctccaatgtctctaaagccaggtgaggaactttcaccaacagatgaaaatggaaaagtaatatttgacatagtggatctctgtaccacctgggaggccatggagaagtgtaaggatgcaggattggccaagtccattggggtgtcaaacttcaaccgcaggcagctggagatgatcctcaacaagccaggactcaagtacaagcctgtctgcaaccaggtagaatgtcatccgtatttcaaccggagtaaattgctagatttctgcaagtcgaaagatattgttctggttgcctatagtgctctgggatctcaacgagacaaacgatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtttttgagttccagttgactgcagaggacatgaaagccatagatggcctagacagaaatctccactattttaacagtgatagttttgctagccaccctaattatccatattcagatgaatatgactacaaagaccatgacggtgattataaagatcatgatatcgattacaaggatgacgatgacaagtaactcgaggggccacggtacccgtatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggga

SEQ ID NO: 3 - DNA, 24 bp, synthetic construct
  source 1..24: mol_type = other DNA
tcagattcttttataggggacaca

SEQ ID NO: 4 - DNA, 24 bp, synthetic construct
  source 1..24: mol_type = other DNA
taaaggccactcaatgctcactaa

SEQ ID NO: 5 - DNA, 20 bp, synthetic construct
  source 1..20: mol_type = other DNA
cgggccacaactcctcataa

SEQ ID NO: 6 - DNA, 20 bp, synthetic construct
  source 1..20: mol_type = other DNA
caatgagcagcgcatcagac

SEQ ID NO: 7 - DNA, 20 bp, synthetic construct
  source 1..20: mol_type = other DNA
tgcaaacatcacatgcacac

SEQ ID NO: 8 - DNA, 20 bp, synthetic construct
  source 1..20: mol_type = other DNA
ttggccccttaccataactg

SEQ ID NO: 9 - DNA, 23 bp, synthetic construct
  source 1..23: mol_type = other DNA
gaagcagaagcttaggaagatgg

SEQ ID NO: 10 - DNA, 24 bp, synthetic construct
  source 1..24: mol_type = other DNA
tgggttgggtgtctgtttcattgt

SEQ ID NO: 11 - DNA, 22 bp, synthetic construct
  source 1..22: mol_type = other DNA
gatccacctgtctctgccttcc

SEQ ID NO: 12 - DNA, 24 bp, synthetic construct
  source 1..24: mol_type = other DNA
gaccttgcattcctttggcgagag

(19) United States
(12) Patent Application Publication
(10) Pub. No.: US 2026/0027232 A1
(43) Pub. Date: Jan. 29, 2026

(54) USE OF AKR1C3 INHIBITOR IN PREPARATION OF DRUG FOR TREATING OR PREVENTING METABOLIC ASSOCIATED FATTY LIVER DISEASE

(71) Applicant: SHENGJING HOSPITAL OF CHINA MEDICAL UNIVERSITY, Liaoning (CN)

(72) Inventors: Yang LIU, Liaoning (CN); Siyu SUN, Liaoning (CN)

(73) Assignee: SHENGJING HOSPITAL OF CHINA MEDICAL UNIVERSITY, Liaoning (CN)

(21) Appl. No.: 18/996,481

(22) PCT Filed: Nov. 24, 2023
(86) PCT No.: PCT/CN2023/133963

§ 371 Date: Jan. 17, 2025

(30) Foreign Application Priority Data
Sep. 30, 2022 (CN) 202211207421.2
Sep. 6, 2023 (CN) 202311144697.5

(51) Int. Cl. A61K 48/00; A01K 67/02; A01K 67/0278; A61K 31/7105; A61K 45/06; A61P 3/06; C07K 16/00; C12N 9/22; C12N 15/113

(52) U.S. Cl. CPC A61K 48/005; A01K 67/02; A01K 67/0278; A61K 31/7105; A61K 45/06; A61P 3/06; C07K 16/00; C12N 9/226; C12N 15/113; A01K 2227/105; A01K 2267/0362; C12N 2310/20

(57) ABSTRACT

Disclosed in the present application are the use of an Aldo-Keto reductase family 1 member C3 (AKR1C3) inhibitor in the inhibition of lipid droplet generation and the promotion of lipid droplet degradation and in the preparation of a drug for treating or preventing MAFLD, a pharmaceutical composition containing the inhibitor, the use of the inhibitor as a drug for treating diseases associated with an abnormal increase in lipid droplets, a method for inhibiting Aldo-Keto reductase family 1 member C3 (AKR1C3), a method for constructing a gene engineering animal model with MAFLD, etc.

### CROSS-REFERENCE TO RELATED APPLICATIONS

The present application claims priority to CN202211207421.2 filed on Sep. 30, 2022 and CN2023111446975 filed on Sep. 6, 2023, the contents of which are incorporated herein by reference in their entirety.

### TECHNICAL FIELD

The present application relates to the field of medicine and pharmaceutical technology, and in particular to the use of an aldo-keto reductase family 1 member C3 (AKR1C3) inhibitor in the preparation of drugs for treating or preventing metabolic associated fatty liver disease.

### BACKGROUND

The liver is an important organ for human lipid metabolism, and is mainly involved in the synthesis and transport of endogenous lipids. Hepatocytes synthesize substances such as triglycerides, phospholipids, and cholesterol by ingesting fatty acids from the circulating blood, quickly converting them into low-density lipoproteins which are then re-excreted into the circulating blood. Lipid synthesis and decomposition in the liver are in a state of dynamic equilibrium. If the lipids accumulated within hepatocytes cannot be metabolized and cleared in time, excessive triglycerides and other toxic lipids tend to accumulate in the cells, leading to steatosis and development of MAFLD, which further induces liver fibrosis and inflammatory hepatocellular damage, forming steatohepatitis, in severe cases, liver cirrhosis and even liver cancer.

Metabolic associated fatty liver disease (MAFLD) is the hepatic manifestation of metabolic syndrome (MetS). The key to its development lies in the initial lipid accumulation and subsequent inflammatory responses. MAFLD is not only an independent risk factor for cardiovascular disease onset, but also increases the risk of death, including the causes of hepatic and non-hepatic diseases, such as diabetes, malignancies, and coronary artery disease, etc. According to statistics, the global per capita prevalence of metabolic associated fatty liver disease (MAFLD) is about 25%, and the prevalence rate in China is as high as 29.4%, and showing an increasing and younger trend year by year. Currently, MAFLD has become the second most common cause of liver transplantation, with about 15-25% of MAFLD cases progressing to non-alcoholic steatohepatitis (NASH), and 20% of NASH patients eventually progressing to cirrhosis or liver cancer. In addition to intrahepatic lesions, extensive clinical evidence shows that MAFLD is independently associated with the development of cardiovascular disease, chronic kidney disease and type 2 diabetes, and can lead to serious multisystem diseases. MAFLD has become the focus of attention in the field of chronic diseases around the world. It is more meaningful to study the mechanism of MAFLD occurrence and to make timely interventions. However, so far, the pathogenesis of MAFLD remains unclear, and no specific drug has been approved for marketing.

In summary, in-depth exploration and research on the pathogenesis of MAFLD and finding potential intervention targets have important social significance and medical research value for the treatment of MAFLD. At the same time, the establishment of a MAFLD transgenic mouse model is of great significance for the drug development of MAFLD.

Aldo-keto reductase family 1 member C3 (AKR1C3) is a member of the oxidoreductase superfamily, and its main function is to catalyze the reduction of aldehydes and ketones to corresponding alcohols using NADH or NADPH as a cofactor, playing an important role in the biosynthesis of prostaglandins and sex hormones. It regulates the metabolism of androgens, estrogens and progesterone, and maintains cell growth and survival.

### SUMMARY

Addressing the above issues, the present application verifies from multiple perspectives such as clinical samples, cell experiments and animal experiments, and reveals for the first time the relationship between AKR1C3 and MAFLD, and targeted inhibition of AKR1C3 can reverse the occurrence and development of MAFLD. It is expected to provide a new theoretical basis and new drug treatment targets for future MAFLD treatment, filling the gap in effective therapeutic drugs for metabolic-associated fatty liver disease. At the same time, the present application also establishes a standardized and reliable MAFLD animal model that can be used for preclinical evaluation of drug efficacy.

In order to achieve the above objectives, the present application provides the following technical solutions:

1. Use of an aldo-keto reductase family 1 member C3 (AKR1C3) inhibitor in inhibiting lipid droplet formation and promoting lipid droplet degradation.

2. The use according to item 1, wherein the AKR1C3 inhibitor comprise at least one of the following I-IV:
I. sgRNA of CRISPR-Cas9 targeting AKR1C3 gene;
II. microRNA, shmiR, siRNA or shRNA targeting AKR1C3 mRNA;
III. an expression vector or another type of vector comprising a sequence of the targeted inhibitor sgRNA, microRNA, shmiR, siRNA or shRNA described in I and II; preferably, the expression vector is one or more selected from the group consisting of: a plasmid expression vector, a retrovirus (RV), a lentivirus (LV), an adenovirus (AV), an adeno-associated virus (AAV), a baculovirus (BV), and a self-replicating virus; preferably, the other type of vector is one or more selected from the group consisting of: a liposome, a polymer nanoparticle, and an RNA nanosphere; and
IV. a targeted protein degrader for targeting AKR1C3 protein homeostasis, wherein the targeted protein degrader is selected from the group consisting of a proteolytic chimera (PROTAC), a molecular glue, a bifunctional degrader, a CHAMP, a lysosomal targeting chimera (LYTAC), a GlueTAC, an antibody-based PROTAC (AbTAC), an autophagy targeting chimera (AUTAC), an ATTEC, and an AUTOTAC.

3. The use according to item 2, wherein the AKR1C3 inhibitor inhibits the AKR1C3 gene of a subject by one or more gene editing technologies selected from the group consisting of: zinc finger, transcription activator-like effector nuclease (TALENS), base editor, prime editor, and AAV directed homology recombination.

4. Use of an aldo-keto reductase family 1 member C3 (AKR1C3) inhibitor in the preparation of a drug for treating or preventing MAFLD.

5. The use according to item 4, wherein the AKR1C3 inhibitor comprises at least one of the following I-IV:
I. sgRNA of CRISPR-Cas9 targeting AKR1C3 gene;
II. microRNA, shmiR, siRNA or shRNA targeting AKR1C3 mRNA;
III. an expression vector or another type of vector comprising a sequence of the targeted inhibitor sgRNA, microRNA, shmiR, siRNA or shRNA described in I and II; preferably, the expression vector is one or more selected from the group consisting of: a plasmid expression vector, a retrovirus (RV), a lentivirus (LV), an adenovirus (AV), an adeno-associated virus (AAV), a baculovirus (BV), and a self-replicating virus; preferably, the other type of vector is one or more selected from the group consisting of: a liposome, a polymer nanoparticle, and an RNA nanosphere; and
IV. a targeted protein degrader for targeting AKR1C3 protein homeostasis, such as a proteolytic chimera (PROTAC), a molecular glue, a bifunctional degrader, a CHAMP, a lysosomal targeting chimera (LYTAC), a GlueTAC, an antibody-based PROTAC (AbTAC), an autophagy targeting chimera (AUTAC), an ATTEC, and an AUTOTAC;
preferably, wherein the gene editing technology used to target AKR1C3 in the above I-III is one or more selected from the group consisting of: zinc finger, transcription activator-like effector nuclease (TALENS), base editor, prime editor, and AAV directed homology recombination.

6. The use according to item 4 or 5, wherein the MAFLD includes simple non-alcoholic fatty liver disease, non-alcoholic steatohepatitis, and liver fibrosis and/or cirrhosis derived therefrom.

7. A pharmaceutical composition, comprising the aldo-keto reductase family 1 member C3 (AKR1C3) inhibitor according to item 2.

8. The pharmaceutical composition according to item 7, wherein the pharmaceutical composition is used to treat or prevent a disease associated with increased lipid droplets.

9. The pharmaceutical composition according to item 8, wherein the disease associated with increased lipid droplets includes MAFLD.

10. The use according to item 9, wherein the MAFLD includes simple non-alcoholic fatty liver disease, non-alcoholic steatohepatitis, and liver fibrosis and/or cirrhosis derived therefrom.

11. Use of an aldo-keto reductase family 1 member C3 (AKR1C3) as a therapeutic target for a disease associated with increased lipid droplets.

12. The use according to item 11, wherein the use of an aldo-keto reductase family 1 member C3 (AKR1C3) as a therapeutic target for a disease associated with increased lipid droplets involves the use of AKR1C3 inhibitor in the preparation of a drug for treating or preventing a disease associated with increased lipid droplets.

13. The use according to item 11 or 12, wherein the disease associated with increased lipid droplets is MAFLD.

14. The use according to claim 13, wherein the MAFLD includes simple non-alcoholic fatty liver disease, non-alcoholic steatohepatitis, and liver fibrosis and/or cirrhosis derived therefrom.

15. The use according to any one of items 12 to 14, wherein the inhibitor comprises at least one of the following I-IV:
I. sgRNA of CRISPR-Cas9 targeting AKR1C3 gene;
II. microRNA, shmiR, siRNA or shRNA targeting AKR1C3 mRNA;
III. an expression vector or another type of vector comprising a sequence of the targeted inhibitor sgRNA, microRNA, shmiR, siRNA or shRNA described in I and II; preferably, the expression vector is one or more selected from the group consisting of: a plasmid expression vector, a retrovirus (RV), a lentivirus (LV), an adenovirus (AV), an adeno-associated virus (AAV), a baculovirus (BV), and a self-replicating virus; preferably, the other type of vector is one or more selected from the group consisting of: a liposome, a polymer nanoparticle, and an RNA nanosphere; and
IV. a targeted protein degrader for targeting AKR1C3 protein homeostasis, such as a proteolytic chimera (PROTAC), a molecular glue, a bifunctional degrader, a CHAMP, a lysosomal targeting chimera (LYTAC), a GlueTAC, an antibody-based PROTAC (AbTAC), an autophagy targeting chimera (AUTAC), an ATTEC, and an AUTOTAC;
preferably, wherein the gene editing technology used to target AKR1C3 in the above I-III is one or more selected from the group consisting of: zinc finger, transcription activator-like effector nuclease (TALENS), base editor, prime editor, and AAV directed homology recombination.

16. A method for inhibiting aldo-keto reductase family 1 member C3 (AKR1C3), comprising interfering with the expression of AKR1C3 protein at the gene level, or interfering with the AKR1C3 protein homeostasis to promote its degradation at the protein level.

17. Use of a vector specifically overexpressing aldo-keto reductase family 1 member C3 (AKR1C3) in the preparation of a MAFLD disease model.

18. The use according to item 17, wherein the vector comprises an AKR1C3 overexpression plasmid and a CAG-LSL-AKR1C3-3×flag-WPRE-pA expression cassette.

19. The use according to item 18, wherein the sequence of the CAG-LSL-AKR1C3-3×flag-WPRE-pA expression cassette is shown as SEQ ID NO: 2.

20. The use according to item 17, wherein the model includes an animal model and a cell model.

21. The use according to item 17, wherein the MAFLD includes simple non-alcoholic fatty liver disease, non-alcoholic steatohepatitis, and liver fibrosis and/or cirrhosis derived therefrom.

22. A method for constructing a MAFLD genetically engineered animal model, wherein the MAFLD genetically engineered animal model is obtained by feeding a mouse in which AKR1C3 gene is overexpressed with a normal diet.

23. The method according to item 22, wherein the MAFLD includes simple non-alcoholic fatty liver disease, non-alcoholic steatohepatitis, and liver fibrosis and/or cirrhosis derived therefrom.

24. A method for constructing a Rosa26 site-directed knock-in mouse model capable of conditionally overexpressing the AKR1C3 gene, wherein the method adopts CRISPR/Cas9 technology to insert a CAG-LSL-AKR1C3-3×flag-WPRE-pA expression cassette at the Rosa26 gene site of mouse chromosome 6 by homologous recombination to obtain the Rosa26 site-directed knock-in mouse capable of conditionally overexpressing the AKR1C3 gene.

25. The method according to item 24, wherein the expression cassette is the CAG-LSL-AKR1C3-3×flag-WPRE-pA expression cassette, and the sequence of which is shown as SEQ ID NO: 2.

26. A method for constructing a MAFLD animal model with liver-specific overexpression of AKR1C3, wherein the Rosa26 site-directed knock-in mouse capable of conditionally overexpressing the AKR1C3 gene prepared in item 24 is selected to crossbreed with a liver-specific Cre (Alb-Cre) mouse to obtain a mouse with liver-specific overexpression of AKR1C3.

27. The method according to item 26, wherein the MAFLD includes simple non-alcoholic fatty liver disease, non-alcoholic steatohepatitis, and liver fibrosis and/or cirrhosis derived therefrom.

28. The method according to item 26 or 27, wherein the MAFLD animal model constructed by the method can be fed with normal diet to obtain the MAFLD animal model.

29. The method according to any one of claims 26 to 28, wherein the MAFLD animal model constructed by the method is used for screening drugs for treating MAFLD and related basic research.

30. A method for constructing a MAFLD animal model with systemic AKR1C3 overexpression, wherein the Rosa26 site-directed knock-in mouse capable of conditionally overexpressing the AKR1C3 gene prepared in item 24 or 25 is selected to crossbreed with an embryonic Cre (Dppa3-Cre) mouse to obtain a mouse with systemic AKR1C3 overexpression.

31. The method according to item 30, wherein the MAFLD includes simple non-alcoholic fatty liver disease, non-alcoholic steatohepatitis, and liver fibrosis and/or cirrhosis derived therefrom.

32. The method according to item 30 or 31, wherein the MAFLD animal model constructed by the method is fed with normal diet for 12 months to obtain the MAFLD animal model.

33. The method according to any one of items 30 to 32, wherein the MAFLD animal model constructed by the method is used for screening drugs for treating MAFLD and related basic research.

### TECHNICAL EFFECTS

Currently, there is no drug approved for the prevention and treatment of MAFLD worldwide. This application is the first to demonstrate that AKR1C3 liver-specific transgenic mice can develop a MAFLD phenotype remarkably similar to the pathophysiology of clinical MAFLD under a normal diet. It also demonstrates that targeted inhibition of AKR1C3 can alleviate the occurrence and development of MAFLD in mice. This application reveals the crucial role of AKR1C3 in promoting liver lipid synthesis, and proposes the application of AKR1C3 as a target in the treatment of MAFLD. It provides an experimental basis and implementation means for the treatment of MAFLD, filling the gap in effective therapeutic drugs for MAFLD.

### BRIEF DESCRIPTION OF THE DRAWINGS

FIG. 1 is a schematic diagram of the positive correlation between AKR1C3 expression level and triglyceride content in liver tissue. FIG. 1A is a schematic diagram of AKR1C3 expression levels in normal liver tissue and fatty liver tissue; FIG. 1B is a bar graph of AKR1C3 expression abundance in normal liver tissue and fatty liver tissue; FIG. 1C is a schematic diagram of the relative triglyceride contents in the high AKR1C3 expression group and the low AKR1C3 expression group. All data was expressed as mean±SEM. “*” indicates a significant difference, wherein * indicates p<0.05 and **** indicates p<0.0001.

FIG. 2 is a schematic diagram of the relationship between downregulation of AKR1C3 at the cellular level and the lipid droplet content in cells Wherein FIG. 2A shows the Western Blot results of AKR1C3 protein expression levels in HepG2 cells transfected with si-scramble (control group) and siAKR1C3, respectively; FIG. 2B shows a comparative fluorescence microscope image of lipid droplet contents in HepG2 cells transfected with si-scramble (control group) and siAKR1C3, respectively; FIG. 2C shows a quantitative analysis of the fluorescence intensity of lipid droplets in HepG2 cells after siAKR1C3 inhibited AKR1C3 compared with si-scramble (control group); FIG. 2D shows Western Blot results of AKR1C3 protein expression levels in HepG2 cells transfected with pLKO.1-scramble (control group) and shAKR1C3 plasmids, respectively; FIG. 2E shows a comparative fluorescence microscope image of lipid droplet contents in HepG2 cells transfected with pLKO.1-scramble (control group) and shAKR1C3, respectively; FIG. 2F shows a quantitative analysis of the fluorescence intensities of lipid droplets within the cells depicted in FIG. 2E; FIG. 2G shows the Western Blot results of AKR1C3 protein expression levels in HepG2-Cas9 stable expression cells transfected with Lenti-guide (control group) and Lenti-sgRNA plasmids, respectively; FIG. 2H shows a comparative fluorescence microscope image of lipid droplets contents in HepG2-Cas9 stable expression cells transfected with Lenti-guide (control group) and Lenti-sgRNA plasmids, respectively; FIG. 2I shows a quantitative analysis of the fluorescence intensity of lipid droplets within the cells depicted in FIG. 2H. It can be seen from FIG. 2 that the content of intracellular lipid droplets decreased significantly after inhibiting AKR1C3 by each of the three pathways, indicating that inhibition of AKR1C3 can inhibit the increase of lipid droplets in cells.

FIG. 3 is an agarose gel electrophoretogram for identifying the Rosa26 LSL genotype on the chromosome of transgenic mice. Wherein FIG. 3A is an electrophoretogram of the amplification product using the first set of primer (i.e., primer pair P1 and P2); FIG. 3B is an electrophoretogram of the amplification product using the second set of primer (i.e., primer pair P3 and P4); FIG. 3C is a 3% agarose gel electrophoretogram of DL2000 DNA marker.

FIG. 4 is an agarose gel electrophoretogram for identifying the Cre genotype on chromosomes of transgenic mice with liver-specific overexpression of AKR1C3. Wherein FIG. 4A is a 3% agarose gel electrophoretogram of DL2000 DNA marker; FIG. 4B is an agarose gel electrophoretogram of PCR products amplified using primers P5, P6 and P7.

FIG. 5 is an agarose gel electrophoretogram for identifying the Cre genotype on chromosomes of transgenic mice with systemic AKR1C3 overexpression. Wherein FIG. 5A is a 3% agarose gel electrophoretogram of DL2000 DNA marker; FIG. 5B is an agarose gel electrophoretogram of PCR products amplified using primers P8, P9 and P10.

FIG. 6 is a schematic diagram of phenotypic validation of MAFLD produced by overexpression of AKR1C3 in mice. FIG. 6A is a schematic diagram of HE staining and Oil Red O staining of liver pathological sections of AKR1C3 transgenic mice; FIG. 6B is a schematic diagram of the relative quantification of the fat vacuole content in HE-stained liver pathological sections of AKR1C3 transgenic mice; FIG. 6C is a schematic diagram of the relative quantification of the lipid droplet content in the liver of AKR1C3 transgenic mice stained with Oil Red O (the black part in FIG. 6A);

FIG. 6D is a schematic diagram of the serum triglyceride level of AKR1C3 transgenic mice. All data was expressed as mean±SEM. “*” indicates significant difference, ** indicates p<0.01, and indicates p<0.0001.

FIG. 7 is a schematic diagram of the improvement of the MAFLD phenotype with adeno-associated virus targeting AKR1C3 in mice. Wherein FIG. 7A is a schematic diagram of HE staining and Oil Red O staining of mouse liver pathological sections after injection of adeno-associated virus to target and inhibit AKR1C3 expression in the liver of transgenic mice; FIG. 7B is a schematic diagram of the relative quantification of the fat vacuole content in the HE-stained liver pathological sections of mice after injection of adeno-associated virus to target and inhibit AKR1C3 expression in the liver of transgenic mice; FIG. 7C is a schematic diagram of the relative quantification of the lipid droplet content in the mice liver stained with Oil Red O (black part in FIG. 7A) after injection of adeno-associated virus to target and inhibit AKR1C3 expression in the liver of transgenic mice; FIG. 7D is a schematic diagram of the serum triglyceride content of mice after injection of adeno-associated virus to target and inhibit AKR1C3 expression in the liver of transgenic mice. All data was expressed as mean±SEM. “*” indicates significant difference, *** indicates p<0.001, and **** indicates p<0.0001.

### DETAIL DESCRIPTION

The present application is described in detail below in conjunction with the embodiments described in the drawings, wherein the same numbers in all drawings represent the same features. Although the drawings depict specific embodiments of the present application, it should be understood that the present application can be implemented in various forms and should not be limited by the embodiments described herein. On the contrary, these embodiments are provided to facilitate a deeper understanding of the present application and to fully convey its scope to those skilled in the art.

It should be noted that certain terminologies are used in the specification and claims to refer to specific components. Those skilled in the art should understand that technicians may use different terms to refer to the same component. This specification and claims do not use the difference in terms as a way to distinguish components, but use the differences in functionality of the components as the criterion for distinguishing. As mentioned throughout the specification and claims, the terms “including” or “comprising” are open-ended terms and should be interpreted as “including but not limited to”. The specification is subsequently described as the preferred embodiment for the present application. However, the description is intended for the general principles of the specification and is not intended to limit the scope of the present application. The scope of protection of the present application shall be determined by the attached claims.

The aldo-keto reductase family 1 member C3 (AKR1C3) refers to the third member within the C subfamily of the aldo-keto reductase family 1, which belongs to a monomeric cytoplasmic protein comprising 323 amino acids, and is about 37 KDa in molecular size. AKR1C3 is ubiquitous in the biological world, and its main function is to reduce aldehydes and ketones to their corresponding alcohols using NADH or NADPH as a cofactor. AKR1C3 also plays an important role in the biosynthesis of prostaglandins and sex hormones.

In one aspect, the present application relates to use of an aldo-keto reductase family 1 member C3 (AKR1C3) inhibitor in inhibiting lipid droplet formation and promoting lipid droplet degradation.

The method for inhibiting aldo-keto reductase family 1 member C3 (AKR1C3) comprises interfering with the expression of AKR1C3 protein at the gene level, or interfering with the AKR1C3 protein homeostasis to promote its degradation at the protein level.

The preparation targeting AKR1C3 refers to a substance that has an inhibitory effect on the expression, activity and stability of AKR1C3 protein. Inhibiting the expression of AKR1C3 protein and promoting the degradation of AKR1C3 protein can effectively inhibit fat formation at the cellular level and in animals.

The preparation targeting/inhibiting AKR1C3 comprises at least one of the following I-IV:
I. sgRNA of CRISPR-Cas9 targeting AKR1C3 gene;
II. microRNA, shmiR (short hairpin microRNA), siRNA or shRNA (short hairpin RNA) targeting AKR1C3 mRNA;
III. an expression vector or another type of vector comprising a sequence of the targeted inhibitor sgRNA, microRNA, shmiR, siRNA or shRNA described in I and II: preferably; the expression vector is one or more selected from the group consisting of: a plasmid expression vector, a retrovirus (RV), a lentivirus (LV), an adenovirus (AV), an adeno-associated virus (AAV), a baculovirus (BV), and a self-replicating virus: preferably, the other type of vector is one or more selected from the group consisting of: a liposome, a polymer nanoparticle, and an RNA nanosphere; and
IV. a targeted protein degrader for targeting AKR1C3 protein homeostasis, such as a proteolytic chimera (PROTAC), a molecular glue, a bifunctional degrader, a CHAMP, a lysosomal targeting chimera (LYTAC), a GlueTAC, an antibody-based PROTAC (AbTAC), an autophagy targeting chimera (AUTAC), an ATTEC, and an AUTOTAC,
preferably, wherein the gene editing technology used to target AKR1C3 in the above I-III is one or more selected from the group consisting of: zinc finger, transcription activator-like effector nuclease (TALENS), base editor, prime editor, and AAV directed homology recombination.

The metabolic associated fatty liver disease (MAFLD) of the present application includes simple non-alcoholic fatty liver disease, non-alcoholic steatohepatitis, and liver fibrosis and/or cirrhosis derived therefrom. Simple non-alcoholic fatty liver disease refers to a condition characterized by increased fat in the liver but little or no inflammation or hepatocellular damage. Non-alcoholic steatohepatitis (NASH) refers to a disease caused by inflammation due to excessive accumulation of fat in the liver. Although subjects with only non-alcoholic fatty liver disease are usually asymptomatic, the inflammation and hepatocellular damage caused by NASH may lead to liver fibrosis or scarring, and in severe cases may lead to cirrhosis (advanced scarring) or liver cancer.

The applicant has conducted a detailed investigation into the relevant mechanisms of metabolic-associated fatty liver disease (MAFLD). After long-term exploration and experimental verification, it was found that aldo-Keto reductase family 1 member C3 (AKR1C3) plays an important regulatory role in hepatic lipid metabolism. According to the results of clinical studies, the expression level of AKR1C3 in fatty liver is significantly higher than that in normal liver tissue. At the same time, comprehensive and in-depth study of lipid metabolism in hepatocytes revealed that AKR1C3 is related to lipid synthesis and lipid droplet content. Therefore, it was hypothesized that inhibiting AKR1C3 can downregulate the fat (lipid droplet) content in hepatocytes, thereby treating MAFLD. According to the above hypothesis, siRNA, shRNA and CRISPR-Cas9 technologies were used to knock down or knock out the AKR1C3 protein level in hepatocytes and reducing AKR1C3 expression. The results show that reduction in AKR1C3 expression results in a significant decrease in intracellular lipids (lipid droplet), indicating that inhibiting AKR1C3 effectively reduces lipid (lipid droplets) accumulation.

In another aspect, the present application relates to use of an aldo-keto reductase family 1 member C3 (AKR1C3) inhibitor in the preparation of a drug for treating or preventing MAFLD.

The AKR1C3 inhibitor includes at least one of the following I-IV:
I. sgRNA of CRISPR-Cas9 targeting AKR1C3 gene;
II. microRNA, shmiR, siRNA or shRNA targeting AKR1C3 mRNA;
III. an expression vector or another type of vector comprising a sequence of the targeted inhibitor sgRNA, microRNA, shmiR, siRNA or shRNA described in I and II: preferably, the expression vector is one or more vectors selected from the group consisting of: a plasmid expression vector, a retrovirus (RV), a lentivirus (LV), an adenovirus (AV), an adeno-associated virus (AAV), a baculovirus (BV), and a self-replicating virus: preferably, the other type of vector is one or more selected from the group consisting of: a liposome, a polymer nanoparticle, and an RNA nanosphere; and
IV. a targeted protein degrader for targeting AKR1C3 protein homeostasis, such as a proteolytic chimera (PROTAC), a molecular glue, a bifunctional degrader, a CHAMP, a lysosomal targeting chimera (LYTAC), a GlueTAC, an antibody-based PROTAC (AbTAC), an autophagy targeting chimera (AUTAC), an ATTEC, and an AUTOTAC;
preferably, wherein the gene editing technology used to target AKR1C3 in the above I-III is one or more selected from the group consisting of: zinc finger, transcription activator-like effector nuclease (TALENS), base editor, prime editor, and AAV directed homology recombination.

The MAFLD includes simple non-alcoholic fatty liver disease, non-alcoholic steatohepatitis, and liver fibrosis and/or cirrhosis derived therefrom.

In another aspect, the present application relates to a pharmaceutical composition, comprising the aldo-keto reductase family 1 member C3 (AKR1C3) inhibitor as described above.

The pharmaceutical composition is used to treat or prevent a disease associated with increased lipid droplets.

The disease associated with increased lipid droplets includes MAFLD.

The MAFLD includes simple non-alcoholic fatty liver disease, non-alcoholic steatohepatitis, and liver fibrosis and/or cirrhosis derived therefrom.

In another aspect, the present application relates to use of an aldo-keto reductase family 1 member C3 (AKR1C3) as a therapeutic target for a disease associated with increased lipid droplets.

The use of an aldo-keto reductase family 1 member C3 (AKR1C3) as a therapeutic target for a disease associated with increased lipid droplets involves the use of AKR1C3 inhibitor in the preparation of a drug for treating or preventing a disease associated with increased lipid droplets.

The disease associated with increased lipid droplets is MAFLD.

The MAFLD includes simple non-alcoholic fatty liver disease, non-alcoholic steatohepatitis, and liver fibrosis and/or cirrhosis derived therefrom.

The inhibitor comprises at least one of the following I-IV:
I. sgRNA of CRISPR-Cas9 targeting AKR1C3 gene;
II. microRNA, shmiR, siRNA or shRNA targeting AKR1C3 mRNA; and
III. an expression vector or another type of vector comprising a sequence of the targeted inhibitor sgRNA, microRNA, shmiR, siRNA or shRNA described in I and II: preferably, the expression vector is one or more selected from the group consisting of: a plasmid expression vector, a retrovirus (RV), a lentivirus (LV), an adenovirus (AV), an adeno-associated virus (AAV), a baculovirus (BV), and a self-replicating virus: preferably, the other type of vector is one or more selected from the group consisting of: a liposome, a polymer nanoparticle, and an RNA nanosphere;
IV. a targeted protein degrader for targeting AKR1C3 protein homeostasis, such as a proteolytic chimera (PROTAC), a molecular glue, a bifunctional degrader, a CHAMP, a lysosomal targeting chimera (LYTAC), a GlueTAC, an antibody-based PROTAC (AbTAC), an autophagy targeting chimera (AUTAC), an ATTEC, and an AUTOTAC;
preferably, wherein the gene editing technology used to target AKR1C3 in the above I-III is one or more selected from the group consisting of: zinc finger, transcription activator-like effector nuclease (TALENS), base editor, prime editor, and AAV directed homology recombination.

The results of mouse in vivo experiment conducted by the applicant on the effect of targeting AKR1C3 to treat MAFLD showed that AKR1C3 transgenic mice can form a MAFLD model under normal diet. Afterwards, the use of adeno-associated virus AAV-TBGp-shAKR1C3 to target and inhibit the expression of AKR1C3 protein has been confirmed to reverse the occurrence and development of MAFLD, demonstrating its therapeutic effect on MAFLD.

In another aspect, the present application relates to a method for inhibiting aldo-keto reductase family 1 member C3 (AKR1C3), comprising interfering with the expression of AKR1C3 protein at the gene level, or interfering with the AKR1C3 protein homeostasis to promote its degradation at the protein level:
wherein interfering with the expression of AKR1C3 protein at the gene level refers to inhibiting the expression of AKR1C3 gene at the gene level, and the inhibitor for inhibiting the expression of AKR1C3 gene is at least one selected from the group consisting of the following I-III: wherein the degrading agent for promoting the degradation of AKR1C3 protein is at least one selected from the following IV:
I. sgRNA of CRISPR-Cas9 targeting AKR1C3 gene;
II. microRNA, shmiR, siRNA or shRNA targeting AKR1C3 mRNA;
III. an expression vector or another type of vector comprising a sequence of the targeted inhibitor sgRNA, microRNA, shmiR, siRNA or shRNA described in I and II: preferably, the expression vector is one or more selected from the group consisting of: a plasmid expression vector, a retrovirus (RV), a lentivirus (LV), an adenovirus (AV), an adeno-associated virus (AAV), a baculovirus (BV), and a self-replicating virus: preferably, the other type of vector is one or more selected from the group consisting of a liposome, a polymer nanoparticle, and an RNA nanosphere; and
IV. a targeted protein degrader for targeting AKR1C3 protein homeostasis, such as a proteolytic chimera (PROTAC), a molecular glue, a bifunctional degrader, a CHAMP, a lysosomal targeting chimera (LYTAC), a GlueTAC, an antibody-based PROTAC (AbTAC), an autophagy targeting chimera (AUTAC), an ATTEC, and an AUTOTAC;
preferably, wherein the gene editing technology used to target AKR1C3 in the above I-III is one or more selected from the group consisting of: zinc finger, transcription activator-like effector nuclease (TALENS), base editor, prime editor, and AAV directed homology recombination.

In another aspect, the present application relates to use of a vector that specifically overexpresses aldo-keto reductase family 1 member C3 (AKR1C3) in the preparation of a MAFLD disease model:
wherein, the vector comprises an AKR1C3 overexpression plasmid and a CAG-LSL-AKR1C3-3×flag-WPRE-pA expression cassette;
the sequence of the CAG-LSL-AKR1C3-3×flag-WPRE-pA expression cassette is shown as SEQ ID NO: 2;
the model includes an animal model and a cell model;
the MAFLD includes simple non-alcoholic fatty liver disease, non-alcoholic steatohepatitis, and liver fibrosis and/or cirrhosis derived therefrom.

In another aspect, the present application also relates to a method for constructing a Rosa26 site-directed knock-in mouse model that can conditionally overexpress the AKR1C3 gene, wherein the method adopts CRISPR/Cas9 technology to insert a CAG-LSL-AKR1C3-3×flag-WPRE-pA expression cassette at the Rosa26 gene site of mouse chromosome 6 by homologous recombination to obtain the Rosa26 site-directed knock-in mouse that can conditionally overexpress the AKR1C3 gene.

The expression cassette is the CAG-LSL-AKR1C3-3×flag-WPRE-pA expression cassette, the sequence of which is shown as SEQ ID NO: 2.

In another aspect, the present application also relates to a method for constructing a MAFLD animal model with liver-specific overexpression of AKR1C3, wherein in the method, the above prepared Rosa26 site-specific knock-in mouse that can conditionally overexpress the AKR1C3 gene is selected to crossbreed with a liver-specific Cre (Alb-Cre) mouse to obtain a mouse with AKR1C3 liver-specific overexpression.

The MAFLD includes simple non-alcoholic fatty liver disease, non-alcoholic steatohepatitis, and liver fibrosis and/or cirrhosis derived therefrom.

The MAFLD animal model with AKR1C3 liver-overexpression constructed by the method can be developed into a MAFLD animal model by feeding with normal diet.

The MAFLD animal model constructed by the method can be used to screen drugs for treating MAFLD.

In another aspect, the present application also relates to a method for constructing a MAFLD animal model with systemic AKR1C3 overexpression, wherein in the method, the above prepared Rosa26 site-specific knock-in mouse that can conditionally overexpress the AKR1C3 gene is selected to crossbreed with an embryonic Cre (Dppa3-Cre) mouse to obtain a mouse with systemic AKR1C3 overexpression.

The MAFLD includes simple non-alcoholic fatty liver disease, non-alcoholic steatohepatitis, and liver fibrosis and/or cirrhosis derived therefrom.

The MAFLD animal model constructed by the method can be fed with normal diet to obtain a MAFLD animal model.

The MAFLD animal model with systemic AKR1C3 overexpression constructed by the method can be used to screen drugs for treating MAFLD.

### EXAMPLE

The present application provides a general and/or specific description of the materials and test methods used in the experiments. In the following examples, unless otherwise specified, % means wt %, i.e., weight percentage. The reagents or instruments used without indicating the manufacturer are all commercially available conventional reagents and products.

### Material:

(1) Cells: HepG2 cells (ATCC).

(2) Plasmid and siRNA:

PsPAX2 plasmid (12260, Addgene),

pMD2.G plasmid (12259, Addgene),

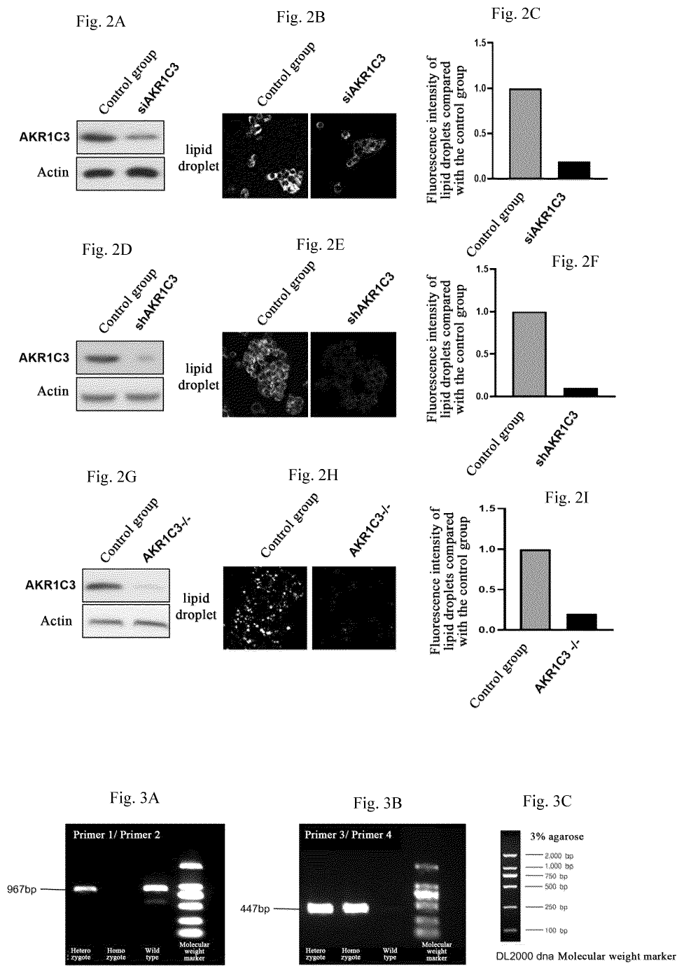

AKR1C3 gene silencing plasmid (pLKO.1-shRNA, labeled as “shAKR1C3” in FIG. 2)

(TRCN0000026561, Sigma),

AKR1C3 gene silencing negative control plasmid (pLKO.1-scramble, labeled as “control group” in FIG. 2) (SCH001, Sigma), note: scramble is a meaningless disordered sequence, which is used as a control.

Cas9 plasmid (52962, Addgene),

AKR1C3 gene knockout plasmid (Lenti-sgRNA, labeled as “AKR1C3−/−” in FIG. 2) (wherein sgRNA was designed using the GPP Web Portal (https://portals.broadinstitute.org/gpp/public/) and synthesized by GENEWIZ, and then sgRNA was ligated to the “Lenti-guide” empty vector to synthesize the AKR1C3 gene knockout plasmid),

AKR1C3 gene knockout negative control plasmid (Lenti-guide empty vector, labeled as “control group” in FIG. 2) (52963, Addgene),

AKR1C3 siRNA (labeled as “siAKR1C3” in FIG. 2) (customized by Jima Biotechnology), siRNA negative control (labeled as “control group” in FIG. 2) (A06001, Jima Biotechnology).

(3) Reagents: DMEM medium (12430062, Gibco), fetal bovine serum (10100147, Gibco), pancreatin (25300054, Gibco), PBS (10010002, Gibco), puromycin dihydrochloride (HY-B1743A, MCE), blasticidin S hydrochloride (S7419, Selleck), cell lysis buffer (R0100, Solarbio), protein quantification kit (23225, ThermoFisher), 5× Loading Buffer (1610767, Bio-red), SDS-PAGE gel preparation kit (P1200, Solarbio), 5× Tris-glycine electrophoresis buffer (T1070, Solarbio), 10× electrophoresis transfer buffer (D1060, Solarbio), 5% BSA blocking solution (SW3015, Solarbio), AKR1C3 antibody (PA5-28065, ThermoFisher), Actin antibody (sc-47778, Santa Cruz), goat anti-rabbit secondary antibody (31786, ThermoFisher), goat anti-mouse secondary antibody (31786, MAI-10378, ThermoFisher), TBST (T1081, Solarbio), ECL luminescent solution (32209, ThermoFisher), Bodipy lipid droplet staining kit (D3922, ThermoFisher), antifade mounting medium (S2100, Solarbio), Oil Red O powder (00625-25G, SIGMA), triglyceride quantitative detection kit (MAK266, sigma), 4% tissue cell fixative (Solarbio), immunohistochemistry UltraSenstive SP kit (KIT-9720, MXB), methanol, isopropanol, 75% ethanol, etc.

(4) Instruments: PVDF transfer membrane (03010040001, Roach), transfer device (1704071, Bio-red), T25 culture flask (707003, NEST), T75 culture flask (708003, NEST), 6 cm culture dish (705001, NEST), 10 cm culture dish (704004, NEST), 6-well plate (7003001, NEST), 15 mL centrifuge tube (601001, NEST), 50 mL centrifuge tube (602001, NEST), 6-well plate cell slide (YA0352, Solarbio), mouse sampling equipment, etc.

### Example 1: The Expression Level of AKR1C3 in Liver Tissue is Positively Correlated with the Content of Triglyceride

Tissue samples from 10 cases of fatty liver were collected (1 case of normal liver tissue, 3 cases of obesity combined with fatty liver, 3 cases of polycystic ovary syndrome combined with fatty liver, and 3 cases of type 2 diabetes combined with fatty liver). The liver tissues were fixed in 4% paraformaldehyde for more than 24 hours. 24 hours later, following gradient alcohol dehydration, xylene permeation, and paraffin embedding, paraffin sections (thickness 4 μm) were prepared for AKR1C3 immunohistochemical staining. The difference in AKR1C3 expression levels between fatty liver and normal liver tissues was compared by immunohistochemistry and quantitatively analyzed.

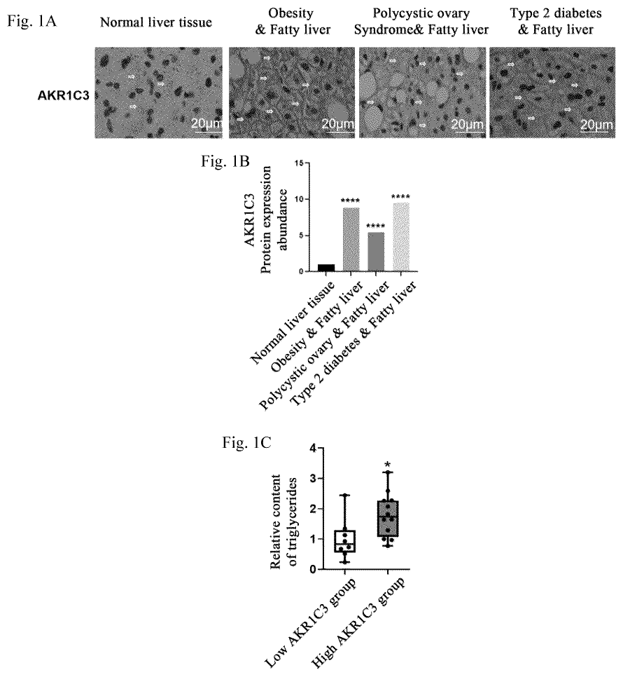

20 liver cancer tissue samples were collected, and were divided into two groups: high AKR1C3 expression group and low AKR1C3 expression group. According to the content of AKR1C3 in normal liver tissue, liver tumor tissues were classified. Tumor tissues with AKR1C3 expression levels lower than that of normal liver tissue constitute the AKR1C3 low expression group (low AKR1C3 group), and tumor tissues with AKR1C3 expression levels higher than that of normal liver tissue constitute the AKR1C3 high expression group (high AKR1C3 group). The differences in triglyceride content between the two groups of tissue samples were detected by liquid chromatography-mass spectrometry and quantitatively analyzed. The above results are shown in FIG. 1: wherein FIG. 1A is a schematic diagram of AKR1C3 expression levels in normal liver tissue and fatty liver tissue: FIG. 1B is a schematic diagram of AKR1C3 expression abundance in normal liver tissue and fatty liver tissue: FIG. 1C is a schematic diagram of the relative triglyceride contents in the high AKR1C3 expression group and the low AKR1C3 expression group. All data was expressed as mean±SEM. * indicates a significant difference, * indicates p<0.05, and **** indicates p<0.0001. As can be seen from FIGS. 1A and 1B, AKR1C3 expression in fatty liver tissue is significantly increased compared with normal liver tissue. As can be seen from FIG. 1C, the content of triglyceride in tumor tissues from high AKR1C3 expression group is higher. This shows that the expression level of AKR1C3 in liver tissue is positively correlated with the content of triglycerides.

### Example 2: Inhibition of AKR1C3 at the Cellular Level can Reduce the Increase in Intracellular Lipid Droplets

The Cas9 plasmid, PsPAX2 plasmid and pMD2.G plasmid were co-transfected into 293T cells using Lipo3000 transfection reagent, allowing for packaging into a virus in 293T cells and subsequent release into the culture medium. After 48 hours, collected the 293T culture medium, and filtered the 293T culture supernatant containing viruses using a 0.45 μm filter membrane to obtain the Cas9 overexpression viral solution, which was then aliquoted and frozen at −80° C. for storage. The Cas9 overexpression viral solution was added to the supernatant of the HepG2 cell culture medium to infect HepG2 cells, and the medium was exchanged after 24 hours. After 48 hours, blasticidin S hydrochloride was added for screening to obtain a HepG2-Cas9) stable expression cell line. Again. 293T cells were selected and transfected simultaneously with Lenti-sgRNA plasmid. PsPAX2 plasmid and pMD2.G plasmid using Lipo3000 transfection reagent to package them into virus in 293T cells and release them into culture medium. After 48 hours, collected the supernatant of the 293T culture medium. and filtered the 293T culture medium containing viruses using a 0.45 μm filter membrane to obtain the AKR1C3 gene knockout viral solution, which was then aliquoted and frozen at −80° C. for storage. The AKR1C3 gene knockout viral solution was added to the supernatant of the HepG2-Cas9) stable expression cell culture medium. and the medium was changed after 24 hours. and puromycin dihydrochloride was added 48 hours later to screen stably transfected cells. Then HepG2 cells with AKR1C3 gene completely knocked out (AKR1C3−/−) were obtained by monoclonal cell line selection. (The sgRNA sequence used by CRISPR-Cas9 is: SEQ ID NO: 1: AATGAGCAGAATCTATATGG). In this example, the inhibition process can be performed either with lentivirus infection comprising Cas9/sgRNA or liposomes comprising siRNA/shRNA to mediate transfection of siRNA/shRNA. A portion of the transfected cells was used to extract total protein, and the inhibition efficiency of AKR1C3 was detected by Western blot: another portion of the transfected cells was plated on a 6-well plate cell slide at 500.000 cells/well. and lipid droplets were stained after the cells were adherent. The specific steps are as follows: aspirated the supernatant of the culture medium, washed the 6-well plate three times with PBS. and prepared the dye solution according to the Bodipy dye instructions, and the incubated at 37° C. in the dark for 20 minutes. Discarded the dye solution, and washed three times with PBS. Prepared an object slide in advance. 10 μL of antifade mounting medium was dropwise added to the surface of the object slide. Removed the cell slide from the 6-well plate and covered it on top of the antifade mounting medium. The intracellular fluorescence content was detected using confocal microscopy.

The above results are shown in FIG. 2, wherein FIG. 2A shows the Western Blot results of AKR1C3 protein expression levels in HepG2 cells transfected with si-scramble (control group) and siAKR1C3. respectively: FIG. 2B shows a comparative fluorescence microscope image of lipid droplet contents in HepG2 cells transfected with si-scramble (control group) and siAKR1C3. respectively: FIG. 2C shows a quantitative analysis of the fluorescence intensity of lipid droplets in HepG2 cells after siAKR1C3 inhibited AKR1C3 compared with si-scramble (control group): FIG. 2D shows Western Blot results of AKR1C3 protein expression levels in HepG2 cells transfected with pLKO.1-scramble (control group) and shAKR1C3 plasmids. respectively: FIG. 2E shows a comparative fluorescence microscope image of lipid droplet contents in HepG2 cells transfected with pLKO.1-scramble (control group) and shAKR1C3 plasmids, respectively: FIG. 2F shows a quantitative analysis of the fluorescence intensities of lipid droplets within the cells depicted in FIG. 2E: FIG. 2G shows the Western Blot results of AKR1C3 protein expression levels in HepG2-Cas9 stable expression cells transfected with Lenti-guide (control group) and Lenti-sgRNA plasmids, respectively: FIG. 2H shows a comparative fluorescence microscope image of lipid droplet contents in HepG2-Cas9 stable expression cells transfected with Lenti-guide (control group) and Lenti-sgRNA plasmids, respectively: FIG. 2I shows a quantitative analysis of the fluorescence intensity of lipid droplets within the cells depicted in FIG. 2H.

As can be seen from FIG. 2, the content of intracellular lipid droplets decreased significantly after inhibiting AKR1C3 by each of the three pathways, indicating that inhibition of AKR1C3 can inhibit the increase of lipid droplets in cells.

### Example 3: Construction of AKR1C3 Liver-Specific Overexpression Mice and AKR1C3 Systemic Overexpression Mice

Note: In the present application, “Alb” (albumin) indicates that the gene-labeled cell type is liver parenchymal cells (to achieve the specific expression of the target gene AKR1C3 in the liver); Dppa3 (developmental pluripotency-associated 3) indicates that the gene-labeled cell type is embryonic germ cells (to achieve systemic expression of the target gene AKR1C3).

AKR1C3^{fl/fl}: fl/fl means “flox/flox”, and AKR1C3^{fl/fl} has the same meaning as Rosa26^{LSL/LSL}, i.e., the “CAG-LSL-AKR1C3-3×flag-WPRE-pA” expression cassette, which comprises a loxP sequence that can be recognized by the Cre recombinase, was inserted at the Rosa26 gene site on both strands of mouse chromosome 6.

CRE^{Alb}: the “IRES-iCre-WPRE-pA” expression cassette was inserted at the stop codon of the mouse Alb gene to enable liver-specific expression of the CRE recombinase.

CRED^{ppa3}: the “IRES-Cre” expression cassette was inserted into the 3′UTR region of the Dppa3 gene. Dppa3-Cre can effectively exert Cre recombinase activity in early embryos and germ cell lines, achieving the purpose of overexpression of Cre recombinase in systemic tissue cells.

The meanings of “Rosa26^{LSL/LSL}: CRE^{Alb}” and “Rosa26^{LSL/LSL}: CRE^{Dppa3}” are the same as those of “AKR1C3^{fl/fl}/CRE^{Alb}” and “AKR1C3^{fl/fl}/CRE^{Dppa3}”.

LSL is the abbreviation of “loxP-stop-loxP” structure. The intracellular Cre recombinase acts on the LSL structure of the “CAG-LSL-AKR1C3-3×flag-WPRE-pA” sequence to activate AKR1C3 expression.

“Rosa26^{LSL/LSL}:Cre^{Alb}” and “AKR1C3^{fl/fl}/CRE^{Alb}”. there are “CAG-LSL-AKR1C3-3×flag-WPRE-pA” expression cassettes on the complementary double strands of DNA of the Rosa26 gene, and the mouse possess the sequence “IRES-iCre-WPRE-pA” on the Alb gene, that is, the mouse has liver-specific overexpression of Cre recombinase, and the Cre recombinase can act on the “CAG-LSL-AKR1C3-3×flag-WPRE-pA” sequence of the Rosa26 gene to achieve liver-specific overexpression of AKR1C3 protein.

“Rosa26^{LSL/LSL}:Cre^{Dppa3}” and “AKR1C3^{fl/fl}/CRE^{Dppa3}”. “CAG-LSL-AKR1C3-3×flag-WPRE-pA” sequence is present on the complementary double strands of DNA of the Rosa26 gene, and there is an “IRES-Cre” expression cassette in the 3′UTR region of the mouse Dppa3 gene, that is, the mouse exhibits systemic overexpression of Cre recombinase in its tissue cells, allowing Cre recombinase to act on the “CAG-LSL-AKR1C3-3×flag-WPRE-pA” sequence of the Rosa26 gene to achieve systemic AKR1C3 protein overexpression.

The meanings of the above abbreviations apply throughout the entire application.

Construction of a Rosa26 site-specific knock-in mouse model that can conditionally overexpress AKR1C3 gene: CRISPR/Cas9 technology was used to insert the CAG-LSL-AKR1C3-3×flag-WPRE-pA expression cassette at the Rosa26 gene site of mouse chromosome 6 by homologous recombination to obtain the Rosa26 site-specific knock-in mouse that can conditionally overexpress AKR1C3 gene. The brief process is as follows: Cas9 mRNA and gRNA were obtained by in vitro transcription: the homologous recombination vector (donor vector), which comprises 3.3 kb of 5′ homology arm, CAG-LSL-AKR1C3-3×flag-WPRE-pA and 3.3 kb of 3′ homology arm, was constructed by In-Fusion cloning. The Cas9mRNA, gRNA and donor vector were microinjected into the fertilized egg of C57BL/6J mouse to obtain F0-generation mouse. The F0-generation mouse which was identified positive by PCR amplification and sequencing was mated with C57BL/6J mouse to obtain 5 positive F1-generation mice, obtaining Rosa26 site-specific knock-in mice with conditional overexpression of the AKR1C3 gene.

The sequence of the CAG-LSL-AKR1C3-3×flag-WPRE-pA expression cassette is shown as SEQ ID NO: 2:

SEQ ID NO: 2:
ACATAACTTACGGTAAATGGCCCGCCTGGCTGACCGCCCAACGACCCCCGCCCATTGACGTCAATA
ATGACGTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTATTGACG
TCAATGACGGTAAATGGCCCGCCTGGCATTATGCCCAGTACATGACCTTATGGGACTTTCCTACT
TGGCAGTACATCTACGTATTAGTCATCGCTATTACCATGGTCGAGGTGAGCCCCACGTTCTGCTT
CACTCTCCCCATCTCCCCCCCCTCCCCACCCCCAATTTTGTATTTATTTATTTTTTAATTATTTT
GTGCAGCGATGGGGGCGGGGGGGGGGGGGGGGCGCGCGCCAGGCGGGGGGGGCGGGGCGAGGGGC
GGGGCGGGGCGAGGCGGAGAGGTGCGGCGGCAGCCAATCAGAGCGGCGCGCTCCGAAAGTTTCCT
TTTATGGCGAGGCGGCGGCGGCGGCGGCCCTATAAAAAGCGAAGCGCGCGGCGGGCGGGGAGTCG
CTGCGACGCTGCCTTCGCCCCGTGCCCCGCTCCGCCGCCGCCTCGCGCCGCCCGCCCCGGCTCTG
ACTGACCGCGTTACTCCCACAGGTGAGCGGGCGGGACGGCCCTTCTCCTCCGGGCTGTAATTAGC
GCTTGGTTTAATGACGGCTTGTTTCTTTTCTGTGGCTGCGTGAAAGCCTTGAGGGGCTCCGGGAG
GGCCCTTTGTGCGGGGGGAGCGGCTCGGGGGGTGCGTGCGTGTGTGTGTGCGTGGGGAGCGCCGC
GTGCGGCTCCGCGCTGCCCGGCGGCTGTGAGCGCTGCGGGCGCGGCGCGGGGCTTTGTGCGCTCC
GCAGTGTGCGCGAGGGGAGCGCGGCCGGGGGCGGTGCCCCGCGGTGCGGGGGGGGCTGCGAGGGG
AACAAAGGCTGCGTGCGGGGTGTGTGCGTGGGGGGGTGAGCAGGGGGTGTGGGCGCGTCGGTCGG
GCTGCAACCCCCCCTGCACCCCCCTCCCCGAGTTGCTGAGCACGGCCCGGCTTCGGGTGCGGGGC
TCCGTACGGGGCGTGGCGCGGGGCTCGCCGTGCCGGGCGGGGGGTGGCGGCAGGTGGGGGTGCCG
GGCGGGGCGGGGCCGCCTCGGGCCGGGGAGGGCTCGGGGGAGGGGCGCGGCGGCCCCCGGAGCGC
CGGCGGCTGTCGAGGCGCGGCGAGCCGCAGCCATTGCCTTTTATGGTAATCGTGCGAGAGGGCGC
AGGGACTTCCTTTGTCCCAAATCTGTGCGGAGCCGAAATCTGGGAGGCGCCGCCGCACCCCCTCT
AGCGGGCGCGGGGCGAAGCGGTGCGGCGCCGGCAGGAAGGAAATGGGCGGGGAGGGCCTTCGTGC
GTCGCCGCGCCGCCGTCCCCTTCTCCCTCTCCAGCCTCGGGGCTGTCCGCGGGGGGACGGCTGCC
TTCGGGGGGGACGGGGCAGGGCGGGGTTCGGCTTCTGGCGTGTGACCGGCGGCTCTAGAGCCTCT
GCTAACCATGTTCATGCCTTCTTCTTTTTCCTACAGCTCCTGGGCAACGTGCTGGTTATTGTGCT
GTCTCATCATTTTGGCAAAGAATTGATTTGATACCGCGGGCCCTAAACGCGTACCAACGTGAAAA
AATTATTATTCGCATAACTTCGTATAGCATACATTATACGAAGTTATCCTCAGCACCATGGCTAG
CGGCAGCCTCGGAGTTTGAATAGATAGAATAAAATATCTTTATTTTCATTCCATCTGTGTGTTGG
TTTTTTGTGTGAGATCTACGGGTGGCATCCCTGTGACCCCTCCCCAGTGCCTCTCCTGGCCCTGG
AAGTTGCCACTCCAGTGCCCACCAGCCTTGTCCTAATAAAATTAAGTTGCATCATTTTGTCTGAC
TAGGTGTCCTTCTATAATATTATGGGGTGGAGGGGGGTGGTATGGAGCAAGGGGCAAGTTGGGAA
GACAACCTGTAGGGCCTGCGGGGTCTATTGGGAACCAAGCTGGAGTGCAGTGGCACAATCTTGGC
TCACTGCAATCTCCGCCTCCTGGGTTCAAGCGATTCTCCTGCCTCAGGCTCCCGAGTTGTTGGGA
TTCCAGGCATGCATGACCAGGCTCAGCTAATTTTTGTTTTTTTGGTAGAGACGGGGTTTCACCAT
ATTGGCCAGGCTGGTCTCCAACTCCTAATCTCAGGTGATCTACCCACCTTGGCCTCCCAAATTGC
TGGGATTACAGGCGTGAACCACTGCTCCCTTCCCTGTCCTTCTGCCTCAGCTAATTGAGTAGGGG
GGAGGCTAACTGAAACACGGAAGGAGACAATACCGGAAGGAACCCGCGCTATGACGGCAATAAAA
AGACAGAATAAAACGCACGGGTGTTGGGTCGTTTGTTCATAAACGCGGGGTTCGGTCCCAGGGCT
GGCACTCTGTCGATACCCCACCGAGACCCCATTGGGGCCAATACGCCCGCGTTTCTTCCTTTTCC
CCACCCCACCCCCCAAGTTCGGGTGAAGGCCCAGGGCTCGCAGCCAACGTCGGGGCGGCAGGCCG
TGGAATTCGTAAATGAATTTTCTGTATGAGGTCGCGATGAATAAATGAAAGCTTGCAGATCTGCG
ACTCTAGAGGATCTGCGACTCTAGAGGATCATAATCAGCCATACCACATTTGTAGAGGTTTTACT
TGCTTTAAAAAACCTCCCACACCTCCCCCTGAACCTGAAACATAAAATGAATGCAATTGTTGTTG
TTAACTTGTTTATTGCAGCTTATAATGGTTACAAATAAAGCAATAGCATCACAAATTTCACAAAT
AAAGCATTTTTTTCACTGCATTCTAGTTGTGGTTTGTCCAAACTCATCAATGTATCTTATCATGT
CTGGATCTGCGACTCTAGAGGATCATAATCAGCCATACCACATTTGTAGAGGTTTTACTTGCTTT
AAAAAACCTCCCACACCTCCCCCTGAACCTGAAACATAAAATGAATGCAATTGTTGTTGTTAACT
TGTTTATTGCAGCTTATAATGGTTACAAATAAAGCAATAGCATCACAAATTTCACAAATAAAGCA
TTTTTTTCACTGCATTCTAGTTGTGGTTTGTCCAAACTCATCAATGTATCTTATCATGTCTGGAT
CTGCGACTCTAGAGGATCATAATCAGCCATACCACATTTGTAGAGGTTTTACTTGCTTTAAAAAA
CCTCCCACACCTCCCCCTGAACCTGAAACATAAAATGAATGCAATTGTTGTTGTTAACTTGTTTA
TTGCAGCTTATAATGGTTACAAATAAAGCAATAGCATCACAAATTTCACAAATAAAGCATTTTTT
TCACTGCATTCTAGTTGTGGTTTGTCCAAACTCATCAATGTATCTTATCATGTCTGGATCCCCAT
CAAGCTGATCCGGAACCCTTAATATAACTTCGTATAGCATACATTATACGAAGTTATTAGGTCCC
TCGACCTGCAGCCCAAGCTAGATCGAATTCGGCCGGCCTTCACGAGCCGCCACCCTCGAGGCCAC
CATGGATTCCAAACACCAGTGTGTAAAGCTAAATGATGGCCACTTCATGCCTGTATTGGGATTTG
GCACCTATGCACCTCCAGAGGTTCCGAGAAGTAAAGCTTTGGAGGTCACAAAATTAGCAATAGAA
GCTGGGTTCCGCCATATAGATTCTGCTCATTTATACAATAATGAGGAGCAGGTTGGACTGGCCAT
CCGAAGCAAGATTGCAGATGGCAGTGTGAAGAGAGAAGACATATTCTACACTTCAAAGCTTTGGT
CCACTTTTCATCGACCAGAGTTGGTCCGACCAGCCTTGGAAAACTCACTGAAGAAAGCTCAATTG
GACTATGTTGACCTCTATCTTATTCATTCTCCAATGTCTCTAAAGCCAGGTGAGGAACTTTCACC
AACAGATGAAAATGGAAAAGTAATATTTGACATAGTGGATCTCTGTACCACCTGGGAGGCCATGG
AGAAGTGTAAGGATGCAGGATTGGCCAAGTCCATTGGGGTGTCAAACTTCAACCGCAGGCAGCTG
GAGATGATCCTCAACAAGCCAGGACTCAAGTACAAGCCTGTCTGCAACCAGGTAGAATGTCATCC
GTATTTCAACCGGAGTAAATTGCTAGATTTCTGCAAGTCGAAAGATATTGTTCTGGTTGCCTATA
GTGCTCTGGGATCTCAACGAGACAAACGATGGGTGGACCCGAACTCCCCGGTGCTCTTGGAGGAC
CCAGTCCTTTGTGCCTTGGCAAAAAAGCACAAGCGAACCCCAGCCCTGATTGCCCTGCGCTACCA
GCTGCAGCGTGGGGTTGTGGTCCTGGCCAAGAGCTACAATGAGCAGCGCATCAGACAGAACGTGC
AGGTTTTTGAGTTCCAGTTGACTGCAGAGGACATGAAAGCCATAGATGGCCTAGACAGAAATCTC
CACTATTTTAACAGTGATAGTTTTGCTAGCCACCCTAATTATCCATATTCAGATGAATATGACTA
CAAAGACCATGACGGTGATTATAAAGATCATGATATCGATTACAAGGATGACGATGACAAGTAAC
TCGAGGGGCCACGGTACCCGTATCAAGCTTATCGATAATCAACCTCTGGATTACAAAATTTGTGA
AAGATTGACTGGTATTCTTAACTATGTTGCTCCTTTTACGCTATGTGGATACGCTGCTTTAATGC
CTTTGTATCATGCTATTGCTTCCCGTATGGCTTTCATTTTCTCCTCCTTGTATAAATCCTGGTTG
CTGTCTCTTTATGAGGAGTTGTGGCCCGTTGTCAGGCAACGTGGCGTGGTGTGCACTGTGTTTGC
TGACGCAACCCCCACTGGTTGGGGCATTGCCACCACCTGTCAGCTCCTTTCCGGGACTTTCGCTT
TCCCCCTCCCTATTGCCACGGCGGAACTCATCGCCGCCTGCCTTGCCCGCTGCTGGACAGGGGCT
CGGCTGTTGGGCACTGACAATTCCGTGGTGTTGTCGGGGAAATCATCGTCCTTTCCTTGGCTGCT
CGCCTGTGTTGCCACCTGGATTCTGCGCGGGACGTCCTTCTGCTACGTCCCTTCGGCCCTCAATC
CAGCGGACCTTCCTTCCCGCGGCCTGCTGCCGGCTCTGCGGCCTCTTCCGCGTCTTCGCCTTCGC
CCTCAGACGAGTCGGATCTCCCTTTGGGCCGCCTCCCCGCATCGATACCGTCGATCCTGTGCCTT
CTAGTTGCCAGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCAGGCATGCTG
GGGATGCGGTGGGCTCTATGGGA.

I. Construction of F2-Generation AKR1C3 Mice with Liver-Specific Overexpression and AKR1C3 Mice with Systemic Overexpression

The above mentioned Rosa26 site-directed knock-in (Rosa26 LSL/+) mice that could conditionally overexpress AKR1C3 gene were crossbreed with liver-specific Cre (Alb-Cre) mice to obtain AKR1C3 mice with liver-specific overexpression (Rosa26 LSL/+: Cre^{Alb}).

The above mentioned Rosa26 site-directed knock-in mice (Rosa26 LSL/+) that could conditionally overexpress AKR1C3 gene were crossbred with embryonic Cre (Dppa3-Cre) mice to obtain AKR1C3 mice with systemic overexpression (Rosa26 LSL/+: Cre^{Dppa3}).

### II. Identification of the Genotype of F2-Generation Mice

Extraction of mouse genomic DNA.

1. For the identification of the Rosa26 LSL genotype on chromosomes in transgenic mice, two sets of PCR primers are required.

The first set comprises:
forward primer P1 (SEQ ID NO: 3)
and reverse primer P2 (SEQ ID NO: 4)
SEQ ID NO: 3:
TCAGATTCTTTTATAGGGGACACA
SEQ ID NO: 4:
TAAAGGCCACTCAATGCTCACTAA;
The second set comprises:
forward primer P3 (SEQ ID NO: 5)
and reverse primer P4 (SEQ ID NO: 6).
SEQ ID NO: 5:
CGGGCCACAACTCCTCATAA
SEQ ID NO: 6:
CAATGAGCAGCGCATCAGAC;

The reaction system was prepared according to the instruction of the mouse tail detection kit (B40013, Biomake). The reaction conditions are shown in Table 1.

TABLE 1

| Step | Temperature (° C.) | Time | | Number of Cycles |
|---|---|---|---|---|
| 1 | 94 | 3 | minutes | |
| 2 | 98 | 20 | seconds | 35 |
| 3 | 61 | 20 | seconds | |
| 4 | 68 | 3 | minutes | |
| 5 | 68 | 5 | minutes | |

| | | |
|---|---|---|
| 6 | 12 | hold |

The gel electrophoresis detection results for PCR products are shown in FIG. 3 (3% agarose gel electrophoresis).

It can be seen from the results in FIG. 3 that the amplification product of the first set of primers (i.e., primer pair P1 and P2) is 967 bp, and the amplification product of the second set of primers (i.e., primer pair P3 and P4) is 447 bp. If the PCR amplification product yields only the 967 bp fragment, the sample is considered wild type (WT); if there are amplification products at both 967 bp and 447 bp, the sample is heterozygous (Rosa26 LSL/+); if the PCR amplification product yields only the 447 bp fragment, the sample is homozygote (Rosa26 LSL/LSL).

2. For the identification of Cre genotype on chromosomes in transgenic mice with liver-specific overexpression of AKR1C3, the required primers are as follows:

primer P5 (SEQ ID NO: 7):
TGCAAACATCACATGCACAC
primer P6 (SEQ ID NO: 8):
TTGGCCCCTTACCATAACTG
primer P7 (SEQ ID NO:9):
GAAGCAGAAGCTTAGGAAGATGG.

The reaction system is shown in table 2.

TABLE 2

| Reaction ingredients | Volume (μL) |
|---|---|
| ddH₂O | 6.5 |
| 2× Taq Plus Master Mix | 10.0 |
| primer P5 (10 pmol/μL) | 0.5 |
| primer P6 (10 pmol/μL) | 0.5 |
| primer P7 (10 pmol/μL) | 0.5 |
| genome DNA | 2 |
| total volume | 20 |

2× Taq Plus Master Mix(dye plus) (P212, Vazyme)

The reaction conditions are shown in table 3.

TABLE 3

| Step | Temperature (° C.) | Time | | Number of Cycles |
|---|---|---|---|---|
| 1 | 94 | 3 | minutes | |
| 2 | 94 | 30 | seconds | 35 |
| 3 | 60 | 30 | seconds | |
| 4 | 72 | 1 | minute | |
| 5 | 72 | 10 | minutes | |

| | | |
|---|---|---|
| 6 | 12 | hold |

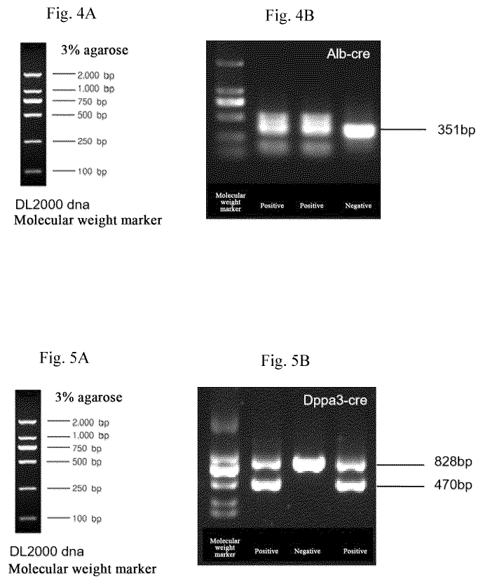

The gel electrophoresis detection results for PCR products are shown in FIG. 4 (3% agarose gel electrophoresis).

In the agarose gel electrophoresis detection results shown in FIG. 4, if the PCR amplification product exhibits only one band at 351 bp, the genotype is Cre-negative; if the PCR amplification product comprises multiple bands, the genotype is Cre-positive.

3. For the identification of Cre genotype on chromosomes in AKR1C3 systemic overexpression transgenic mice, the required primers are as follows:

primer P8 (SEQ ID NO: 10):
TGGGTTGGGTGTCTGTTTCATTGT
primer P9 (SEQ ID NO: 11):
GATCCACCTGTCTCTGCCTTCC
primer P10 (SEQ ID NO: 12):
GACCTTGCATTCCTTTGGCGAGAG

The reaction system is shown in table 4:

TABLE 4

| Reaction ingredient | Volume (μL) |
|---|---|
| ddH₂O | 6.5 |
| 2× Taq Plus Master Mix | 10.0 |
| primer P8 (10 pmol/μL) | 0.5 |
| primer P9(10 pmol/μL) | 0.5 |
| primer P10 (10 pmol/μL) | 0.5 |
| genome DNA | 2 |
| total volume | 20 |

2× Taq Plus Master Mix(dye plus) (P212, Vazyme)

The reaction conditions are shown in table 5:

TABLE 5

| Step | Temperature (° C.) | Time | | Number of Cycles |
|---|---|---|---|---|
| 1 | 94 | 3 | minutes | |
| 2 | 94 | 30 | seconds | 35 |
| 3 | 61 | 30 | seconds | |
| 4 | 72 | 1 | minute | |
| 5 | 72 | 5 | minutes | |

| | | |
|---|---|---|
| 6 | 12 | hold |

The gel electrophoresis detection results for PCR products are shown in FIG. 5 (3% agarose gel electrophoresis).

In the agarose gel electrophoresis detection results shown in FIG. 5, if the PCR amplification product shows only a band at 828 bp, the genotype is Cre-negative; if the PCR amplification product shows bands at both 470 bp and 828 bp, the genotype is Cre-positive heterozygote; if the PCR amplification product shows only a band at 470 bp, the genotype is Cre-positive homozygote.

### III. Breeding of AKR1C3 Transgenic Mice:

8-28 weeks old Rosa26^{LSL/LSL} female (or male) mice were selected and mated with Rosa26^{LSL/LSL}:Cre^{Alb} (or Rosa26^{LSL/LSL}:Cre^{Dppa3}) male (or female) mice, and female mice could give birth after about 21 days of pregnancy. At 10 days after birth, neonatal mice underwent toe clipping for identification purposes and the genotype was detected (method same as above). This breeding method can yield Rosa26^{LSL/LSL}:Cre^{Alb} (or Rosa26^{LSL/LSL}:Cre^{Dppa3}) (50%) mice and Rosa26^{LSL/LSL} (50%) mice.

### Example 4: AKR1C3 Liver-Specific Overexpression in Mice can Produce MAFLD Phenotype

Based on the results of in vitro experiments, it was hypothesized that increased hepatic AKR1C3 levels can promote the increase of liver fat in vivo and thus promote the development of fatty liver.

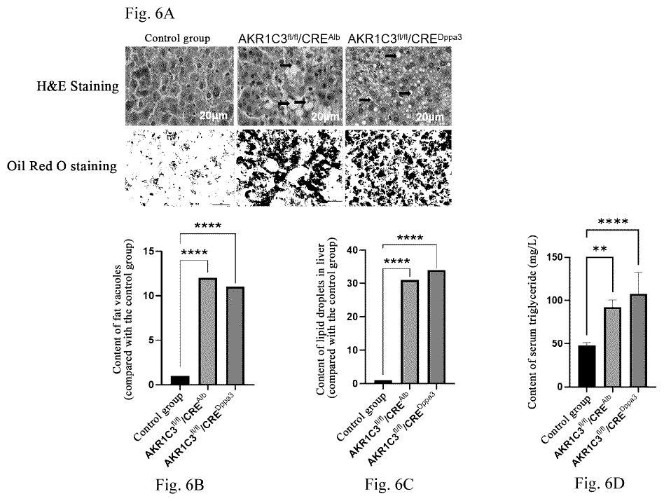

Experimental scheme: AKR1C3 transgenic mice were fed with normal diet (1010082, Synergy Biology), and samples of mice (n=10) were collected at the 12th month. The mice were euthanized, and liver tissue was taken and fixed in 4% paraformaldehyde for more than 24 hours. After 24 hours, a part of the tissue was dehydrated by gradient alcohol, permeabilized by xylene, and embedded in paraffin to make paraffin sections (thickness 4 μm) for H&E staining. Another part of the fixed tissue was placed in 30% sucrose solution overnight. The next day, it was quickly frozen at −80° C., embedded in OCT to prepare frozen pathological sections (thickness 10 μm) for Oil Red O staining. The specific steps were carried out according to the reagent instructions. Meanwhile, whole blood was collected in a 1.5 ml centrifuge tube, left to solidify at room temperature for at least 1 hour, and centrifuged at 4° C. and 3000 rpm for 10 minutes. The content of serum triglyceride was detected using a triglyceride quantification kit (MAK266, sigma). The results are shown in FIG. 6.

FIG. 6A is a schematic diagram of HE staining and Oil Red O staining of liver pathological sections of AKR1C3 transgenic mice: FIG. 6B is a schematic diagram of the relative quantification of the fat vacuole content in HE-stained liver pathological sections of AKR1C3 transgenic mice: FIG. 6C is a schematic diagram of the relative quantification of the lipid droplet content in the liver of AKR1C3 transgenic mice stained with Oil Red O: FIG. 6D is a schematic diagram of the serum triglyceride level of AKR1C3 transgenic mice. All data was expressed as mean±SEM. “*” indicates significant difference. ** indicates p<0.01, and **** indicates p<0.0001.

It can be seen from FIG. 6 that the contents of fat vacuoles (white part in the diagram) and lipid droplets (black part in the diagram) in the liver tissue of AKR1C3 overexpressing mice under normal diet are significantly higher than those in the control group. At the same time, the serum triglyceride level of AKR1C3 overexpressing mice under normal diet was significantly higher than that of the control group. The above results show that under normal diet conditions, AKR1C3 overexpressing in mice leads to hepatic steatosis and increased serum triglycerides, manifesting a fatty liver phenotype.

### Example 5: Targeted Inhibition of AKR1C3 Expression in Mouse Liver with Adeno-Associated Virus can Improve the MAFLD Phenotype

Based on the results of the above in vitro and in vivo experiments, in order to further explore the key role of AKR1C3 in the occurrence and development of MAFLD in vivo, we used adeno-associated virus to target and inhibit the expression of AKR1C3 protein in the liver of transgenic mice, to explore the inhibitory effect of targeted inhibition of AKR1C3 on the occurrence and development of MAFLD.

Construction method of adenovirus: AAV8 serotype was selected. The vector backbone GV681 (purchased from Shanghai Jikai Gene Technology Co., Ltd.). Sequence of the elements is: TBGp-EGFP-MCS-SV40-Ploy A. Restriction sites are NheI and HindIII. The scramble (control sequence) and shAKR1C3 sequences were inserted into the GV681 vector backbone using molecular cloning technology. The recombinant expression plasmid was co-transfected into HEK-293 cells with pHelper (carrying a gene of adenovirus origin) and pAAV-RC (carrying AAV replication and capsid genes). The recombinant AAV packaging was completed 2-3 days after transfection, and the supernatant and cells were collected. The viruses were collected by purification and filtration, and the virus concentration was titrated by quantitative PCR.

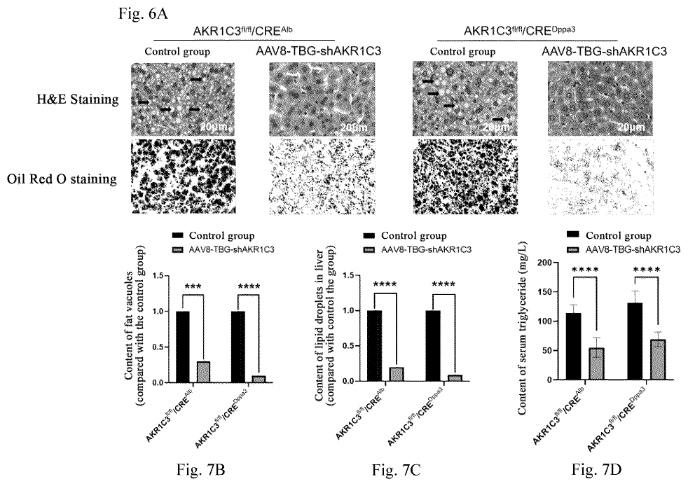

Experimental scheme: 10-month-old AKR1C3^{fl/fl}:Cre^{Alb} and AKR1C3^{fl/fl}:Cre^{Dppa3} mice, totaling 12 for each, were selected and randomly divided into two groups. AAV8-TBG-scramble viral solution (control group) and AAV8-TBG-shAKR1C3 viral solution were injected through the tail vein at a concentration of 5×10¹¹ GC/mouse. The status of the mice was recorded weekly after injection. At the age of 12 months, the mice were euthanized and their serum was collected and stored at −80° C. In subsequent experiments, the serum was used to detect the content of serum triglyceride. The liver tissue was preserved in liquid nitrogen for subsequent testing. The degree of hepatic steatosis in mice was evaluated by H&E staining and Oil Red O staining. The results are shown in FIG. 7.

FIG. 7A is a schematic diagram of HE staining and Oil Red O staining of mouse liver pathological sections after injection of adeno-associated virus to target and inhibit AKR1C3 expression in the liver of transgenic mice: FIG. 7B is a schematic diagram of the relative quantification of the fat vacuole content in the HE-stained liver pathological sections of mice after injection of adeno-associated virus to target and inhibit AKR1C3 expression in the liver of transgenic mice: FIG. 7C is a schematic diagram of the relative quantification of the lipid droplet content in the mice liver stained with Oil Red O (black part in FIG. 7A) after injection of adeno-associated virus to target and inhibit AKR1C3 expression in the liver of transgenic mice: FIG. 7D is a schematic diagram of the serum triglyceride content of mice after injection of adeno-associated virus to target and inhibit AKR1C3 expression in the liver of transgenic mice. All data was expressed as mean±SEM. “*” indicates significant difference, *** indicates p<0.001, and **** indicates p<0.0001.

It can be seen from FIG. 7 that, under normal diet conditions, compared with the control group, the contents of fat vacuoles (white part in the diagram) and lipid droplets (black part in the diagram) in the liver tissue of AKR1C3 transgenic mice injected with AAV8-TBG-shAKR1C3 (i.e., inhibiting AKR1C3 expression) were significantly reduced, and the serum triglyceride level was also reduced. The above results show that inhibiting AKR1C3 in the in vivo environment alleviates the increase of liver fat (reduced fat vacuoles and lipid droplets) and decreases blood lipids, thereby alleviating the occurrence and development of MAFLD.

The foregoing represents only the preferred embodiments of the present application, and does not impose any other forms of limitations on the present application. Any person skilled in the art may use the technical content disclosed above to make changes or modifications to equivalent embodiments with equivalent variations. However, any simple modification, equivalent substitutions and variations made to the above embodiments based on the technical essence of the present application, without departing from the content of the technical solution of the present application, still belongs to the protection scope of the technical solution of the present application.

1-3. (canceled)

4. Use of an aldo-keto reductase family 1 member C3 (AKR1C3) inhibitor in the preparation of a drug for treating or preventing MAFLD.

5. The use according to claim 4, wherein the AKR1C3 inhibitor comprises at least one of the following I-IV:
I. sgRNA of CRISPR-Cas9 targeting AKR1C3 gene;
II. microRNA, shmiR, siRNA or shRNA targeting AKR1C3 mRNA;
III. an expression vector or another type of vector comprising a sequence of the targeted inhibitor sgRNA, microRNA, shmiR, siRNA or shRNA described in I and II; preferably, the expression vector is one or more selected from the group consisting of: a plasmid expression vector, a retrovirus (RV), a lentivirus (LV), an adenovirus (AV), an adeno-associated virus (AAV), a baculovirus (BV), and a self-replicating virus; preferably, the other type of vector is one or more selected from the group consisting of: a liposome, a polymer nanoparticle, and an RNA nanosphere; and
IV. a targeted protein degrader for targeting AKR1C3 protein homeostasis, such as a proteolytic chimera (PROTAC), a molecular glue, a bifunctional degrader, a CHAMP, a lysosomal targeting chimera (LYTAC), a GlueTAC, an antibody-based PROTAC (AbTAC), an autophagy targeting chimera (AUTAC), an ATTEC, and an AUTOTAC;
preferably, wherein the gene editing technology used to target AKR1C3 in the above I-III is one or more selected from the group consisting of: zinc finger, transcription activator-like effector nuclease (TALENS), base editor, prime editor, and AAV directed homology recombination.

6. The use according to claim 4, wherein the MAFLD includes simple non-alcoholic fatty liver disease, non-alcoholic steatohepatitis, and liver fibrosis and/or cirrhosis derived therefrom.

7. A pharmaceutical composition, comprising the aldo-keto reductase family 1 member C3 (AKR1C3) inhibitors according to claim 5.

8. The pharmaceutical composition according to claim 7, wherein the pharmaceutical composition is used to treat or prevent MAFLD.

9. (canceled)

10. The use according to claim 8, wherein the MAFLD includes simple non-alcoholic fatty liver disease, non-alcoholic steatohepatitis, and liver fibrosis and/or cirrhosis derived therefrom.

11. (canceled)

12. Use of an aldo-keto reductase family 1 member C3 (AKR1C3) inhibitor in the preparation of a drug for treating or preventing a disease associated with increased lipid droplets.

13. The use according to claim 12, wherein the disease associated with increased lipid droplets is MAFLD.

14. The use according to claim 13, wherein the MAFLD includes simple non-alcoholic fatty liver disease, non-alcoholic steatohepatitis, and liver fibrosis and/or cirrhosis derived therefrom.

15. The use according to claim 12, wherein the inhibitor comprises at least one of the following I-IV:
I. sgRNA of CRISPR-Cas9 targeting AKR1C3 gene;
II. microRNA, shmiR, siRNA or shRNA targeting AKR1C3 mRNA;
III. an expression vector or another type of vector comprising a sequence of the targeted inhibitor sgRNA, microRNA, shmiR, siRNA or shRNA described in I and II; preferably, the expression vector is one or more selected from the group consisting of: a plasmid expression vector, a retrovirus (RV), a lentivirus (LV), an adenovirus (AV), an adeno-associated virus (AAV), a baculovirus (BV), and a self-replicating virus; preferably, the other type of vector is one or more selected from the group consisting of: a liposome, a polymer nanoparticle, and an RNA nanosphere; and
IV. a targeted protein degrader for targeting AKR1C3 protein homeostasis, such as a proteolytic chimera (PROTAC), a molecular glue, a bifunctional degrader, a CHAMP, a lysosomal targeting chimera (LYTAC), a GlueTAC, an antibody-based PROTAC (AbTAC), an autophagy targeting chimera (AUTAC), an ATTEC, and an AUTOTAC;
preferably, wherein the gene editing technology used to target AKR1C3 in the above I-III is one or more selected from the group consisting of: zinc finger, transcription activator-like effector nuclease (TALENS), base editor, prime editor, and AAV directed homology recombination.

16-23. (canceled)

24. A method for constructing a Rosa26 site-directed knock-in mouse model capable of conditionally overexpressing the AKR1C3 gene, wherein the method adopts CRISPR/Cas9 technology to insert a CAG-LSL-AKR1C3-3×flag-WPRE-pA expression cassette at the Rosa26 gene site of mouse chromosome 6 by homologous recombination to obtain the Rosa26 site-directed knock-in mouse capable of conditionally overexpressing the AKR1C3 gene.

25. The method according to claim 24, wherein the expression cassette is the CAG-LSL-AKR1C3-3×flag-WPRE-pA expression cassette, and the sequence of which is shown as SEQ ID NO: 2.

26. A method for constructing a MAFLD animal model with liver-specific overexpression of AKR1C3, wherein the Rosa26 site-directed knock-in mouse capable of conditionally overexpressing the AKR1C3 gene prepared in claim 24 is selected to crossbreed with a liver-specific Cre (Alb-Cre) mouse to obtain a mouse with liver-specific overexpression of AKR1C3.

27. The method according to claim 26, wherein the MAFLD includes simple non-alcoholic fatty liver disease, non-alcoholic steatohepatitis, and liver fibrosis and/or cirrhosis derived therefrom.

28. The method according to claim 26, wherein the MAFLD animal model constructed by the method can be fed with normal diet to obtain the MAFLD animal model.

29. The method according to claim 26, wherein the MAFLD animal model constructed by the method is used for screening drugs for treating MAFLD and related basic research.

30. A method for constructing a MAFLD animal model with systemic AKR1C3 overexpression, wherein the Rosa26 site-directed knock-in mouse capable of conditionally overexpressing the AKR1C3 gene prepared in claim 24 is selected to crossbreed with an embryonic Cre (Dppa3-Cre) mouse to obtain a mouse with systemic AKR1C3 overexpression.

31. The method according to claim 30, wherein the MAFLD includes simple non-alcoholic fatty liver disease, non-alcoholic steatohepatitis, and liver fibrosis and/or cirrhosis derived therefrom.

32. The method according to claim 30, wherein the MAFLD animal model constructed by the method is fed with normal diet for 12 months to obtain the MAFLD animal model.

33. The method according to claim 30, wherein the MAFLD animal model constructed by the method is used for screening drugs for treating MAFLD and related basic research.
